(19) United States
(12) Patent Application Publication
(10) Pub. No.: US 2018/0339964 A1
(43) Pub. Date: Nov. 29, 2018

(54) THE PROCESS OF PREPARING INDOLINE COMPOUNDS AND A NOVEL INDOLINESALT

(71) Applicant: BIOCON LIMITED, Electronic City P.O. Kamataka (IN)

(72) Inventors: Chandrashekar ASWATHANARAYANAPPA, Gottigere (IN); Thilak Gregory SOUNDARARAJAN, Subramaniapuram (IN); Nooka Appa Rao GORLI V, Gopalapuram Post & Village (IN); Surendra Babu JAGABATHUNI, Chirala (IN); Palle Venkata Raghavendra CHARYULU, Hyderabad (IN)

(21) Appl. No.: 15/763,030

(22) PCT Filed: Sep. 21, 2016
(86) PCT No.: PCT/IB2016/055625

§ 371 Date: Mar. 23, 2018

(30) Foreign Application Priority Data
Sep. 23, 2015 (IN) 5079/CHE/2015
May 19, 2016 (IN) 201641017352

(51) Int. Cl. C07D 209/14; A61K 31/405

(52) U.S. Cl. CPC C07D 209/14; A61K 31/405

(57) ABSTRACT

The present invention provides an industrial method for production of silodosin, which is useful for a therapeutic agent for dysuria associated with benign prostatic hyperplasia. The production of silodosin is characterized by mixing (R)-1-(3-hydroxypropyl)-5-(2-(2-(2-(2, 2, 2-trifluoroethoxy) phenoxy) ethyl amino) propyl) indoline-7-carbonitrile (V) and N-acetyl-L-glutamic acid to yield the N-acetyl-L-glutamate salt, subsequently neutralising the N-acetyl-L-glutamate salt and hydrolyzing the same, and manufacturing intermediates used therefore. The invention also provides an industrial production method of silodosin alpha, beta and gamma crystalline forms.

### RELATED APPLICATION

This application claims the benefit of priority of our Indian patent application numbers 5079/CHE/2015 filed on Sep. 23, 2015 & 201641017352 filed on May 19, 2016 which are incorporated herein by reference.

### TECHNICAL FIELD

The present invention relates to a method for production of the indoline compound and a novel salt of indoline. More particularly the present invention relates to method for production of an indoline compound (general name: Silodosin) represented by the following structural formula

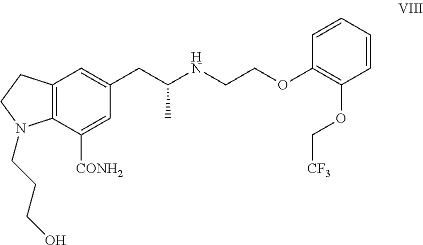 [SMILES: C[C@H](Cc1cc2c(c(C(N)=O)c1)N(CCCO)CC2)NCCOc1ccccc1OCC(F)(F)F]

Which is useful as a therapeutic agent for the symptomatic treatment of benign prostatic hyperplasia and a novel N-acetyl glutamate salt of indoline useful in the production.

### BACKGROUND AND PRIOR ART OF THE DISCLOSURE

Silodosin belongs to a group of drugs called alpha-adrenergic (AL-fa ad-ren-ER-jik) blockers. Silodosin helps relax the muscles in the prostate and bladder neck, making it easier to urinate. Silodosin is used to improve urination in men with benign prostatic hyperplasia (enlarged prostate).

As an effective and efficient method for production of silodosin, it is proposed or reported that an optically active amine compound represented by the following general formula:

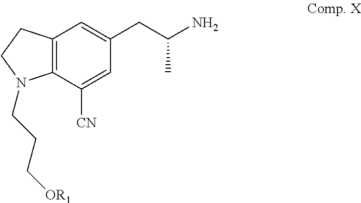

wherein R1 represents a hydrogen atom or a hydroxyl-protective group, is allowed to react with a phenoxyethane compound represented by the following general formula:

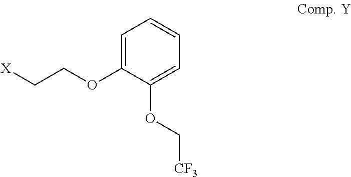

wherein X represents a leaving group, and optionally deprotected and the cyano group is converted to a carbamoyl group (see Patent References 3 and 4).

However, in the above-mentioned methods for production, a dialkyl compound (Comp. Z) represented by the following general formula:

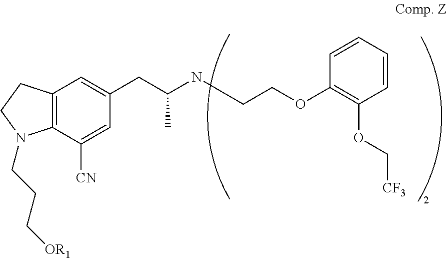

wherein R1 represents a hydrogen atom or a hydroxyl-protective group, is sometime generated as a by-product because of the reaction of one molecule of the optically active amine compound and two molecules of the phenoxyethane compound. Since it is difficult to remove the by-product by purification method used in a common industrial production such as recrystallization or the like, it is necessary to use purification method such as column chromatography or the like to remove the by-product. Therefore purification processes tend to be complex, are not satisfactory a method for industrial production.

Also methods to hydrolyze the 3-{7-cyano-5-[(2R)-2-({2-[2-(2,2,2-trifluoroethoxy)phenoxy]ethyl}amino)propyl]-2,3-dihydro-1H-indol-1-yl}propyl benzoate monooxalate salts to 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile as disclosed in WO 2006/046499 A1 suffered from significant amount of dialkyl impurity (Comp. Z-a and Comp.Z-b)

Polymorphism refers to the occurrence of different crystalline forms of the same drug substance. It includes solvation products and amorphous forms. It is often characterized as the ability of a drug substance to exist as two or more crystalline phases that have different arrangements and/or conformations of the molecules in the crystal lattice.

The formation of different polymorphic forms can be achieved by crystallizing the compound from different solvents under varying conditions. Polymorph formation is influenced by temperature of the solution, rate of stirring, rate of precipitation, mode of mixing and rate of addition of the mixing of solvents and time of stirring. Commonly used techniques for crystallization include solvent evaporation, slow or sudden cooling of the solution, solvent/non-solvent diffusion, anti-solvent, pH shifting, vapor diffusion, sublimation and many variations on these processes.

Silodosin is known to exist in different physical forms referred to as polymorphs.

European patent no. EP 1,541,554 B1 discloses three different crystal forms of silodosin viz., (1) a crystal characterized by main peaks of 5.5°+0.2°, 6.1°±0.2°, 9.8°±0.2°, 11.1°±0.20, 12.2°±0.20, 16.4°±0.20, 19.7°±0.20 and 20.0°±0.2° as 2 θ [hereinafter referred to as crystalline alpha (a) silodosin]; (2) a crystal characterized by main peaks of 7.0°±0.20, 12.5°±0.20, 18.5°±0.20, 19.5°±0.20, 20.7°±0.2° and 21.1°±0.2° as 2 θ [hereinafter referred to as crystalline beta (β) silodosin]; and (3) a crystal characterized by main peaks of 6.0°±0.2°, 10.6°±0.20, 12.6°±0.2°, 17.1°±0.2°, 17.9°±0.2°, 20.7°±0.2° and 23.7°±0.2° as 2 θ [hereinafter referred to as crystalline gamma (γ) silodosin.

EP '554 discloses the crystalline beta silodosin can be prepared by dissolving crude crystals thereof in an appropriate amount of methanol under heating, adding petroleum ether solvent, stirring the mixture vigorously, such that the crystals are forcibly and suddenly precipitated. The crystalline beta silodosin can also be prepared by dissolving crude crystal thereof in ethanol or 1-propanol, and cooling quickly.

CN 103360298 discloses processes for preparation of crystalline beta silodosin by dissolving the crude silodosin in a first solvent selected from the group consisting of chloroform, dichloroethane, dichloromethane and adding a second solvent selected from the group consisting of cyclohexane, isopropyl ether, methyl tert-butyl ether, n-butanone, n-hexane and filtration, cooling the filtrate to 0-5° C. crystallization 0-2 hours, the crystals were collected by filtration, and dried to give crystal form 0 Silodosin.

WO2012147107 discloses process for preparation of crystalline beta silodosin by treating the crude silodosin in isopropyl acetate at 70-75° C. followed by cooling to room temperature followed by filtration. Also a similar process with Methyl isobutyl ketone for preparation of crystalline beta silodosin.

Thus, the development of a more applicable purification method for industrial production is required.

Patent Reference 1: Japanese Patent Publication H6-220015;

Patent Reference 2: Japanese Patent Publication 2000-247998;

Patent Reference 3: Japanese Patent Publication 2001-199956;

Patent Reference 4: Japanese Patent Publication 2002-265444.

Patent Reference 5: U.S. Pat. No. 7,834,193 B2.

The present disclosure aims to provide a method for industrial production of Silodosin.

The present invention provides for a one pot process for the preparation of beta crystalline form of silodosin.

The present invention also provides for the process for the preparation of gamma crystalline form of silodosin which is economical and industrially feasible.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

FIG. 1 shows the XRD of crystalline solid (2R)-1-(7-cyano-1-(3-hydroxypropyl)-2,3-dihydro-1H-inden-5-yl)-N-(2-(2-(2,2,2-trifluoroethoxy)phenoxy)ethyl)propan-2-aminium (R)-4-acetamido-4-carboxybutanoate (Compound VI)

FIG. 2 shows the DSC of crystalline solid (2R)-1-(7-cyano-1-(3-hydroxypropyl)-2,3-dihydro-1H-inden-5-yl)-N-(2-(2-(2,2,2-trifluoroethoxy)phenoxy)ethyl)propan-2-aminium (R)-4-acetamido-4-carboxybutanoate (Compound VI).

FIG. 3 shows the TGA of crystalline solid (2R)-1-(7-cyano-1-(3-hydroxypropyl)-2,3-dihydro-1H-inden-5-yl)-N-(2-(2-(2,2,2-trifluoroethoxy)phenoxy)ethyl)propan-2-aminium (R)-4-acetamido-4-carboxybutanoate (Compound VI).

FIG. 4 shows the XRD of Gamma form of crystalline solid 1-(3-Hydroxypropyl)-5-[(2R)-2-({2-[2-(2,2,2-trifluoroethoxy)phenoxy]ethyl}amino)propyl]-2,3-dihydro-1H-indole-7-carboxamide (Compound VIII).

FIG. 5 shows the XRD of Beta form of crystalline solid 1-(3-Hydroxypropyl)-5-[(2R)-2-({2-[2-(2,2,2-trifluoroethoxy)phenoxy]ethyl}amino)propyl]-2,3-dihydro-1H-indole-7-carboxamide (Compound VIII).

FIG. 6 shows the XRD of Alpha form of crystalline solid 1-(3-Hydroxypropyl)-5-[(2R)-2-({2-[2-(2,2,2-trifluoroethoxy)phenoxy]ethyl}amino)propyl]-2,3-dihydro-1H-indole-7-carboxamide (Compound VIII).

FIG. 7 shows the XRD spectrum (R)-1-(1-(3-(benzoyloxy)propyl)-7-cyanoindolin-5-yl)-N-(2-(2-(2,2,2-trifluoroethoxy)phenoxy)ethyl)propan-2-aminium (S)-4-acetamido-4-carboxybutanoate (IV-a).

The method of analysis of the compounds represented in the figures as above are as below:

### PXRD Analysis

About 300 mg of powder sample was taken onto the sample holder and was tightly packed on the sample holder uniformly by means of glass slide and Powder X-ray diffraction was recorded on Bruker D8 Advance diffractometer (Bruker-AXS, Karlsruhe, Germany) using Cu-Kα X-radiation (λ=1.5406 Å) at 40 kV and 30 mA powder.X-ray diffraction patterns were collected over the 2θ range 3-50° at a scan rate of 1°/min.

### DSC Analysis

DSC was performed on a Mettler Toledo DSC 822e module. 4-6 mg of sample was placed in crimped but vented aluminium sample pans. The temperature range was from 30-250° C. @ 10° C./min. Samples were purged by a stream of nitrogen flowing at 80 mL/min.

### TGA Analysis

TGA was performed on a Mettler Toledo TGA/SDTA 851e module. About 4-6 mg of sample was taken in a ceramic crucible and carefully placed on the balance and weight loss of the sample on gradual heating was measured. The temperature range was from 30-350° C. @ 10° C./min. Samples were purged by a stream of nitrogen flowing at 80 mL/min.

### DETAILED DESCRIPTION OF THE DISCLOSURE

The present disclosure provides that by converting 3-{7-cyano-5-[(2R)-2-({2-[2-(2,2,2-trifluoroethoxy)-phenoxy]ethyl}amino)propyl]-2,3-dihydro-1H-indol-1-yl}-propyl benzoate represented by the following structural formula (IV) and 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile of the structural formula (V):

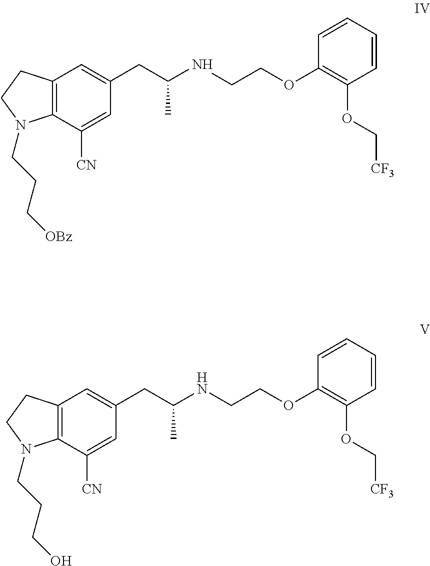

to the N-acetyl glutamate salts and isolating the same by crystallization, the by-product (Comp. Z-a and Comp.Z-b) represented by the formulas:

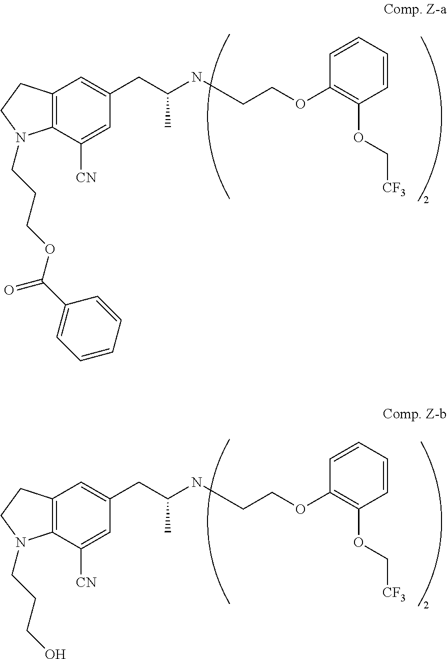

can be removed, thereby forming the basis of the present invention.

That is, the present invention relates to a method for production of 1-(3-hydroxypropyl)-5-[(2R)-2-({2-[2-(2,2,2-trifluoroethoxy)phenoxy]ethyl}amino)propyl]-2,3-dihydro-1H-indole-7-carboxamide represented by the structural formula (VIII):

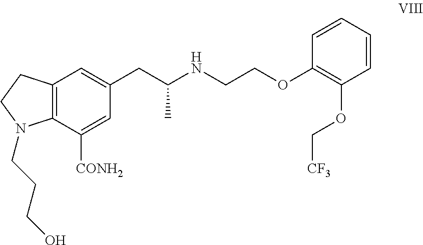 [SMILES: C[C@H](Cc1cc2c(c(C(N)=O)c1)N(CCCO)CC2)NCCOc1ccccc1OCC(F)(F)F]

which comprises mixing (R)-1-(3-hydroxypropyl)-5-(2-(2-(2-(2,2,2-trifluoroethoxy) phenoxy)ethylamino)propyl)indoline-7-carbonitrile represented by the following formula (V):

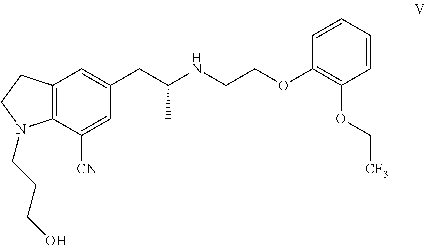 [SMILES: C[C@H](Cc1cc(C#N)c2c(c1)CCN2CCCO)NCCOc1ccccc1OCC(F)(F)F]

with N-acetyl glutamic acid to yield the (R)-1-(3-hydroxypropyl)-5-(2-(2-(2-(2,2,2-trifluoroethoxy)phenoxy)ethylamino)propyl)indoline-7-carbonitrile mono N-acetyl glutamate, subsequently neutralising the N-acetyl glutamate to yield (R)-1-(3-hydroxypropyl)-5-(2-(2-(2-(2,2,2-trifluoroethoxy)phenoxy)ethylamino)propyl)indoline-7-carbonitrile represented by the structural formula (VII):

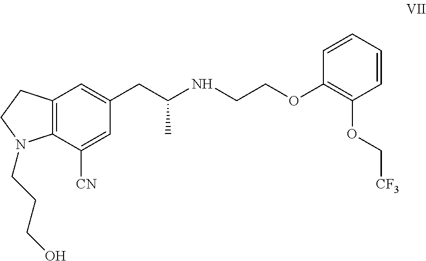 [SMILES: C[C@H](Cc1cc(C#N)c2c(c1)CCN2CCCO)NCCOc1ccccc1OCC(F)(F)F]

and hydrolysing the compound represented by the structural formula (VII), and manufacturing intermediates used in the method for production.

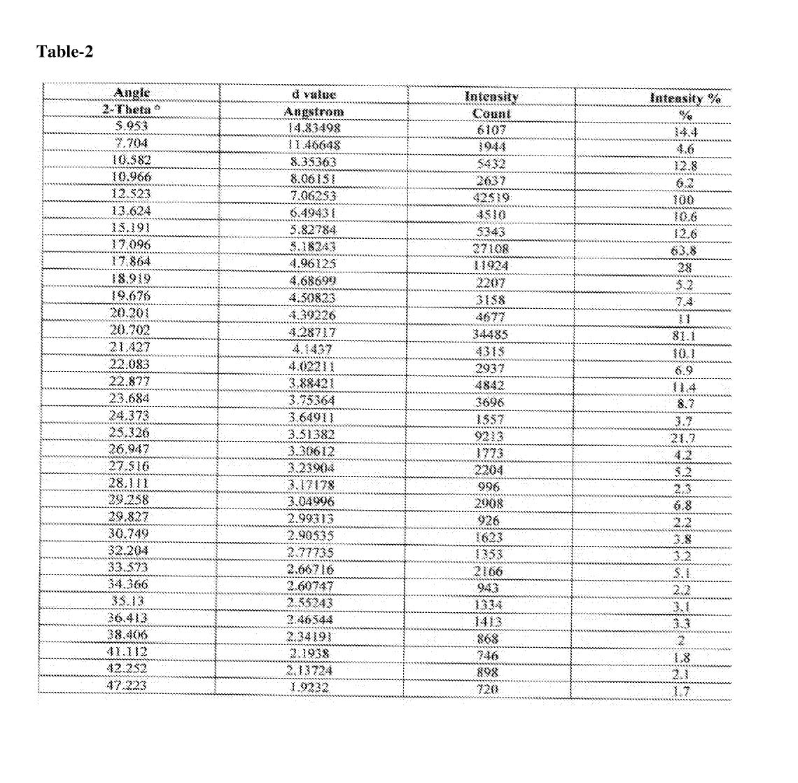

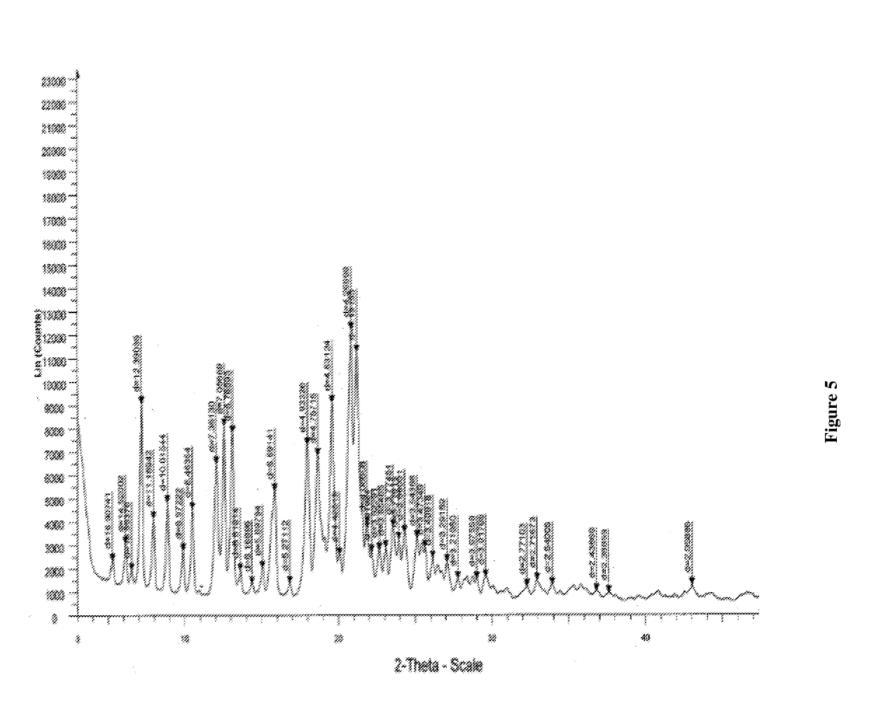

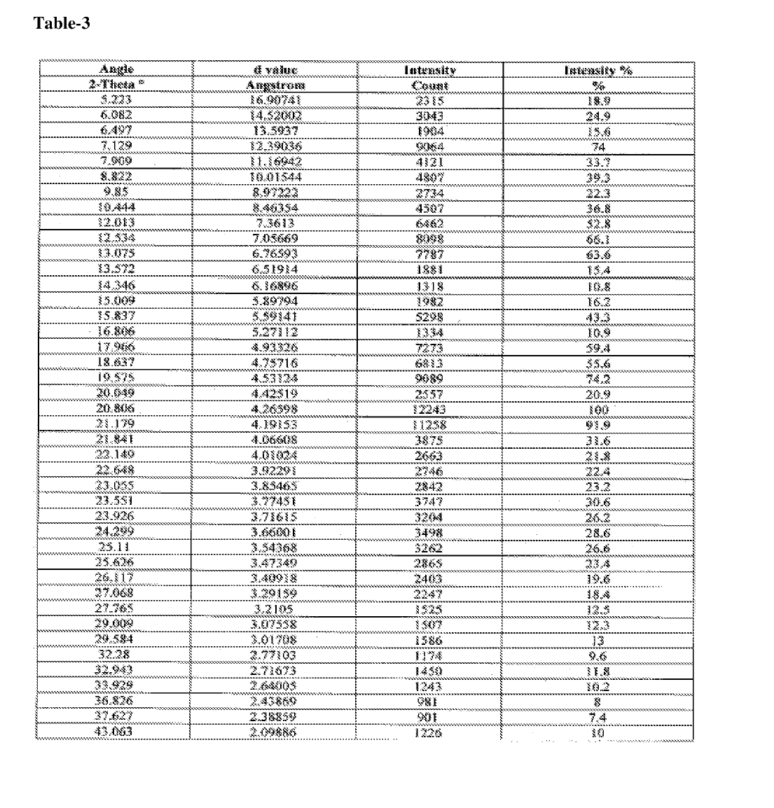

The present invention also provides for the process for preparation of crystalline Gamma form of Silodosin represented by the structural formula (VIII) which is represented in the FIG. 4.

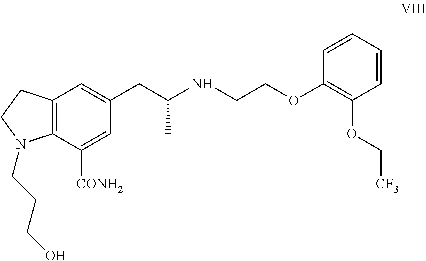 [SMILES: C[C@H](Cc1cc2c(c(C(N)=O)c1)N(CCCO)CC2)NCCOc1ccccc1OCC(F)(F)F]

That is, present invention relates to a process for the production of crystalline Gamma form of Silodosin comprising addition of a C1 to C5 aliphatic alcohol and an aromatic hydrocarbon solvent to the crude silodosin and stirred at 60-90° C. till dissolution and allowing to cool the reaction mass to the room temperature slowly followed by stirring at room temperature for 1-2 h. Adding a C1 to C5 alkyl ether to the reaction mass and stirring at RT for 1-2 h, filtering off the crystallised solid to yield (R)-1-(3-hydroxypropyl)-5-(2-(2-(2-(2,2,2-trifluoroethoxy)phenoxy) ethyl amino)propyl)indoline-7-carboxamide (VIII). Slurrying the wet cake with C1 to C5 alkyl ether and filtering off, drying under vacuum to yield silodosin in crystalline gamma form.

The present invention also provides for a one pot process for production of crystalline Beta form of silodosin.

That is, the present invention relates to a one pot process for production of crystalline beta form of 1-(3-hydroxypropyl)-5-[(2R)-2-({2-[2-(2,2,2-trifluoroethoxy)phenoxy]ethyl}amino) propyl]-2,3-dihydro-1H-indole-7-carboxamide represented by the structural formula (VIII):

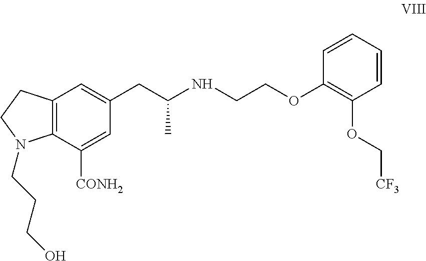 [SMILES: C[C@H](Cc1cc2c(c(C(N)=O)c1)N(CCCO)CC2)NCCOc1ccccc1OCC(F)(F)F]

Which comprises addition of an alkali solution to (R)-1-(3-hydroxypropyl)-5-(2-(2-(2-(2,2,2-trifluoroethoxy)phenoxy)ethyl amino) propyl) indoline-7-carbonitrile (VII) (7.0 g) solution in DMSO at a lower temperature, preferably between 0 to 15° C., warming the reaction mixture to room temperature and stirring for the reaction completion (around 5 to 8 h). Adding a solution of sodium sulphite to the reaction mass and diluting with a C1 to C3 halogenated hydrocarbon solvent. Separating the layers, diluting the organic layer with an aromatic hydrocarbon solvent and concentrated the organic layer at 50 to 65° C. to remove C1 to C3 halogenated hydrocarbon completely and stirred at 70 to 90° C. Adding a C1 to C5 alkyl ether solution to the organic layer stirred it for 30 min, partially cooling the reaction mass to 60±10° C. and seeding with 1% beta silodosin. Stirring the reaction mixture at RT for 1 hr, filtering off and drying under vacuum to yield silodosin in crystalline beta form.

The present invention also provides for a process for production of crystalline Beta form of silodosin from crystalline gamma form of silodosin.

That is, the present invention relates to a process for production of crystalline beta form of 1-(3-hydroxypropyl)-5-[(2R)-2-({2-[2-(2,2,2-trifluoroethoxy)phenoxy]ethyl}amino) propyl]-2,3-dihydro-1H-indole-7-carboxamide represented by the structural formula (VIII):

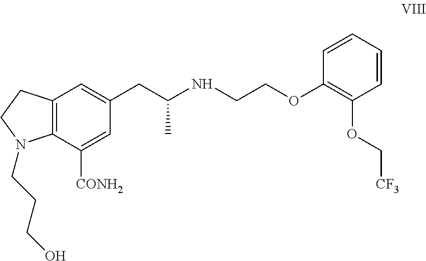 [SMILES: C[C@H](Cc1cc2c(c(C(N)=O)c1)N(CCCO)CC2)NCCOc1ccccc1OCC(F)(F)F]

Which comprises dissolving crystalline gamma form of Silodosin in an aromatic hydrocarbon solvent at an elevated temperature followed by addition of a C1 to C5 alkyl ether solution and stirring for 1-2 h at the same temperature, partial cooling to 60° C. Seeding with 1% Silodosin beta form and stirring for 1 h at RT, filtering off the solid to yield silodosin in crystalline beta form.

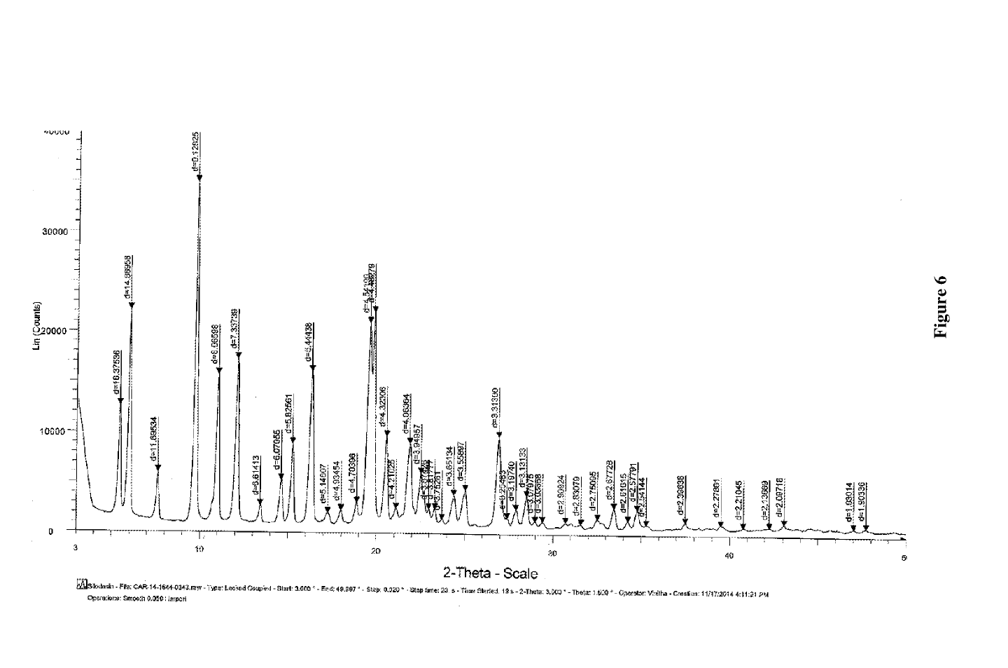

The present invention also provides for the process for preparation of crystalline alpha form of Silodosin represented by the structural formula (VIII) which is represented in the FIG. 6.

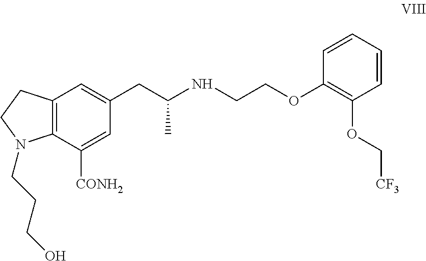 [SMILES: C[C@H](Cc1cc2c(c(C(N)=O)c1)N(CCCO)CC2)NCCOc1ccccc1OCC(F)(F)F]

That is, the present invention relates to a process for production of crystalline alpha form of 1-(3-hydroxypropyl)-5-[(2R)-2-({2-[2-(2,2,2-trifluoroethoxy)phenoxy]ethyl}amino) propyl]-2,3-dihydro-1H-indole-7-carboxamide represented by the structural formula (VIII):

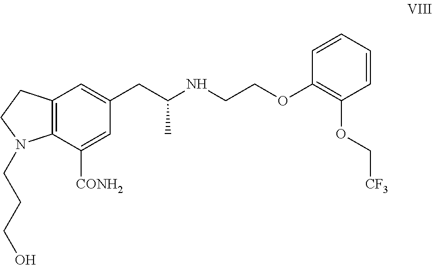 [SMILES: C[C@H](Cc1cc2c(c(C(N)=O)c1)N(CCCO)CC2)NCCOc1ccccc1OCC(F)(F)F]

Comprising dissolving the crude solid (VIII) in an C1 to C4 alkyl ester solvent stirring at an elevated temperature of about 40 to 60° C. for dissolution then stirring at RT, filtering off the solid to yield silodosin in crystalline alpha form.

As discussed herein the term “C1 to C5 aliphatic alcohols” refers to the alcohols selected from methanol, ethanol, propanol, isopropanol, butanol, pentanol and the like; “aromatic hydrocarbon” refers to benzene, toluene, xylene and the like; “C1 to C5 alkyl ethers” refers to dimethyl ether, diethyl ether, methyl ethyl ether, methyl tertiary butyl ether and the like; “alkali solution” refers to the aqueous solution of sodium hydroxide, potassium hydroxide, sodium bicarbonate and the like; “C1 to C3 halogenated hydrocarbon” refers to chloroform, dichloromethane, chloro ethane, dichloroethane and the like; “alkyl ester” refers to methyl acetate, ethyl acetate, ethyl propionate and the like.

### Effect of the Invention

(R)-1-(3-hydroxypropyl)-5-(2-(2-(2-(2,2,2-trifluoroethoxy)phenoxy)ethylamino)propyl) indoline-7-carbonitrile mono N-acetyl glutamate generated as an intermediate in the method for production of the present invention crystallizes well, is easy to separate from the by-product (Comp. Z-b) and easy to handle. Therefore, this N-acetyl glutamate salt is an excellent intermediate in the method for industrial production.

### EXAMPLES

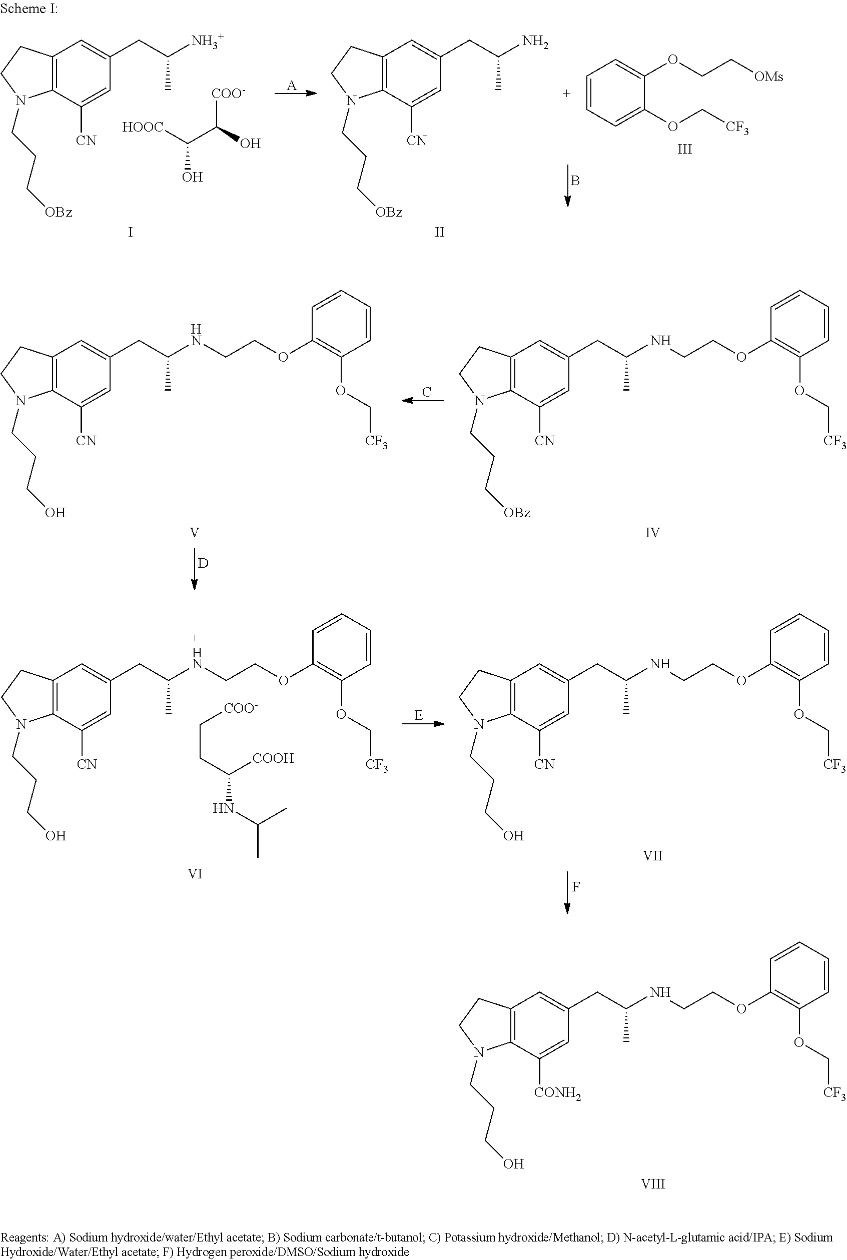

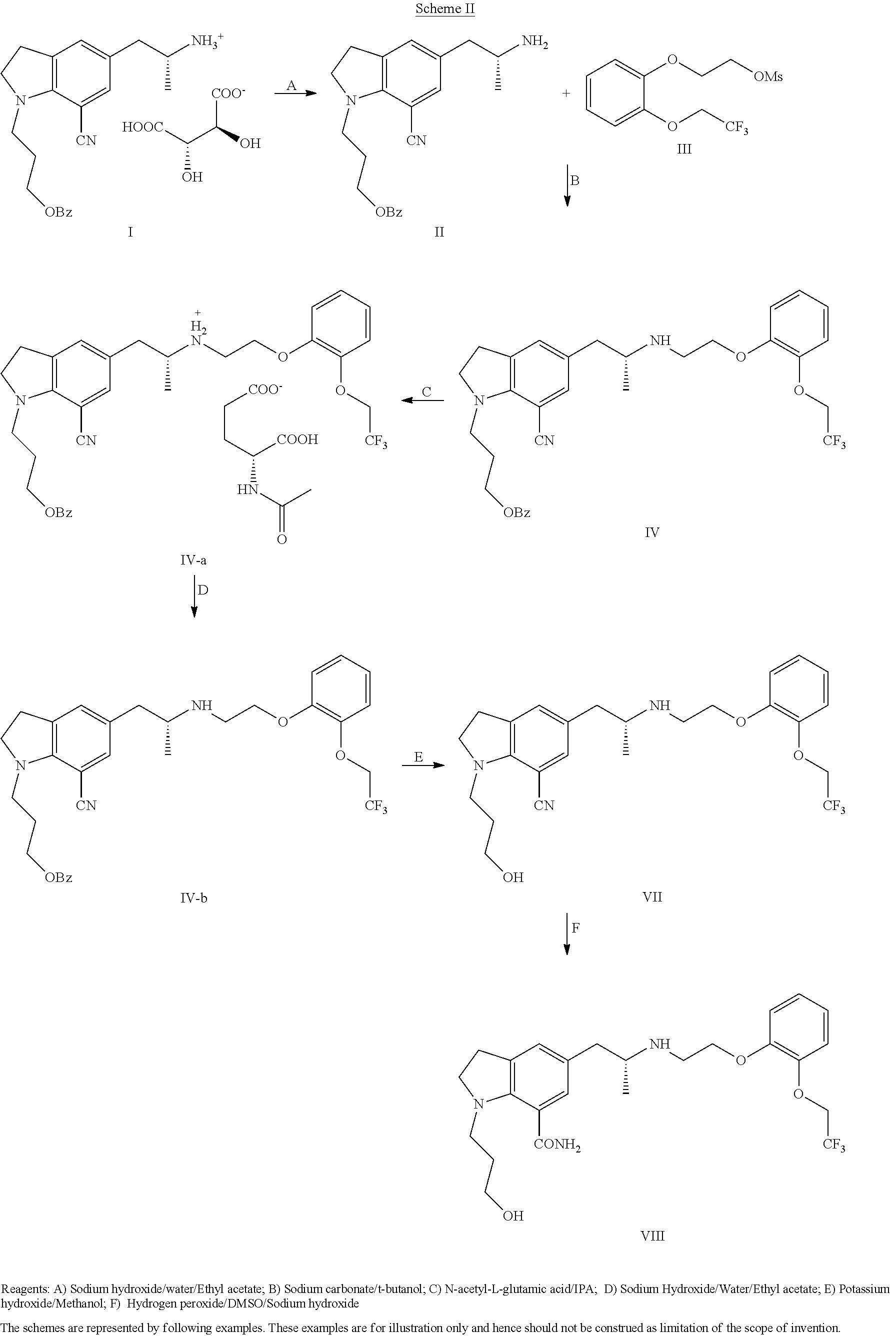

### Example 1: Preparation of (R)-3-(5-(2-aminopropyl)-7-cyanoindolin-1-yl) propyl benzoate (II)

(R)-1-(1-(3-(benzoyloxy)propyl)-7-cyanoindolin-5-yl)propan-2-aminium (2S,3S)-3-carboxy-2,3-dihydroxypropanoate (I) (5.0 kg) was dissolved in Water (25 L) and Ethyl acetate (40 L) and basified with 25% sodium hydroxide solution to adjust to pH-10, layers were separated, organic layer was washed with water followed by brine solution, organic layer was concentrated up to thick syrup to yield (R)-3-(5-(2-aminopropyl)-7-cyanoindolin-1-yl) propyl benzoate (II) (3.5 kg).

### Example—2: Preparation of (R)-3-(7-cyano-5-(2-(2-(2-(2,2,2-trifluoroethoxy)phenoxy) ethylamino)propyl)indolin-1-yl)propyl benzoate (IV)

(R)-3-(5-(2-aminopropyl)-7-cyanoindolin-1-yl)propyl benzoate (II) (3.5 kg) was dissolved in t-BuOH (50 L), added Na₂CO₃ (1.12 eq.,) to the mass, stirred for 10 min, added 2-(2-(2, 2, 2-trifluoroethoxy) phenoxy) ethyl methane sulfonate (III) (1.5 eq.,) to the reaction mass, stirred the reaction mass for reaction completion at 81° C. around 45 to 50 hrs. Water (25 L) and Toluene (25 L) was added to the reaction mass and stirred it for 10 min, layers were separated and organic layer was washed with bicarbonate solution, followed by brine solution. Organic layer was concentrated completely to thick syrup, (R)-3-(7-cyano-5-(2-(2-(2-(2,2,2-trifluoroethoxy)phenoxy)ethylamino)propyl)indolin-1-yl)propyl benzoate (IV) (4.6 kg syrup).

### Example—3: Preparation of (R)-1-(3-hydroxypropyl)-5-(2-(2-(2-(2,2,2-trifluoroethoxy) phenoxy)ethylamino)propyl)indoline-7-carbonitrile (V)

(R)-3-(7-cyano-5-(2-(2-(2-(2,2,2-trifluoroethoxy)phenoxy)ethylamino)propyl)indolin-1-yl)propyl benzoate (IV) (11.5 g) was dissolved in Methanol (75 mL), potassium hydroxide (4 eq.) solution (dissolve in water) was added to the reaction mixture and stirred at 65° C. for reaction completion (around 5 hrs). The reaction mass was concentrated completely to remove Methanol. Water (75 mL) and DCM (75 mL) was added and stirred for 10 min, layers were separated, organic layer was washed with bicarbonate solution and brine solution, organic layer was concentrated completely to (R)-1-(3-hydroxypropyl)-5-(2-(2-(2-(2,2,2-trifluoroethoxy) phenoxy)ethylamino)propyl)indoline-7-carbonitrile (V) (9.5 g) as thick syrup.

### Example—4: Preparation of (2R)-1-(7-cyano-1-(3-hydroxypropyl)-2,3-dihydro-1H-inden-5-yl)-N-(2-(2-(2,2,2-trifluoroethoxy)phenoxy)ethyl)propan-2-aminium (R)-4-acetamido-4-carboxybutanoate (VI)

(R)-1-(3-hydroxypropyl)-5-(2-(2-(2-(2,2,2-trifluoroethoxy)phenoxy)ethylamino)propyl) indoline-7-carbonitrile (V) (9.5 g syrup) was dissolved in IPA (100 mL), N-acetyl-L-glutamic acid (1.0 eq.,) was added to it and the reaction mass was stirred at 25° C. for 5 h and then at 5 to 10° C. for 1 hr, the solid was filtered off and dried at 40° C. under vacuum to (2R)-1-(7-cyano-1-(3-hydroxypropyl)-2,3-dihydro-1H-inden-5-yl)-N-(2-(2-(2,2,2-trifluoroethoxy)phenoxy) ethyl) propan-2-aminium(R)-4-acetamido-4-carboxybutanoate (VI) (10.5 g).

1H-NMR (DMSO-d6) δ ppm: 1.0 (6H, d, J=6.1 Hz), 1.8 (6H, m), 2.2 (2H, t), 2.4 (1H, dd), 2.7 (1H, dd), 2.9 (2H, t), 3.1 (2H, m), 3.5 (4H, m), 3.7 (1H, m, J=6.7 Hz), 4.1 (2H, m), 4.7 (2H, q, J=9.8 Hz), 6.9-7.1 (5H, m), 7.9 (1H, d).

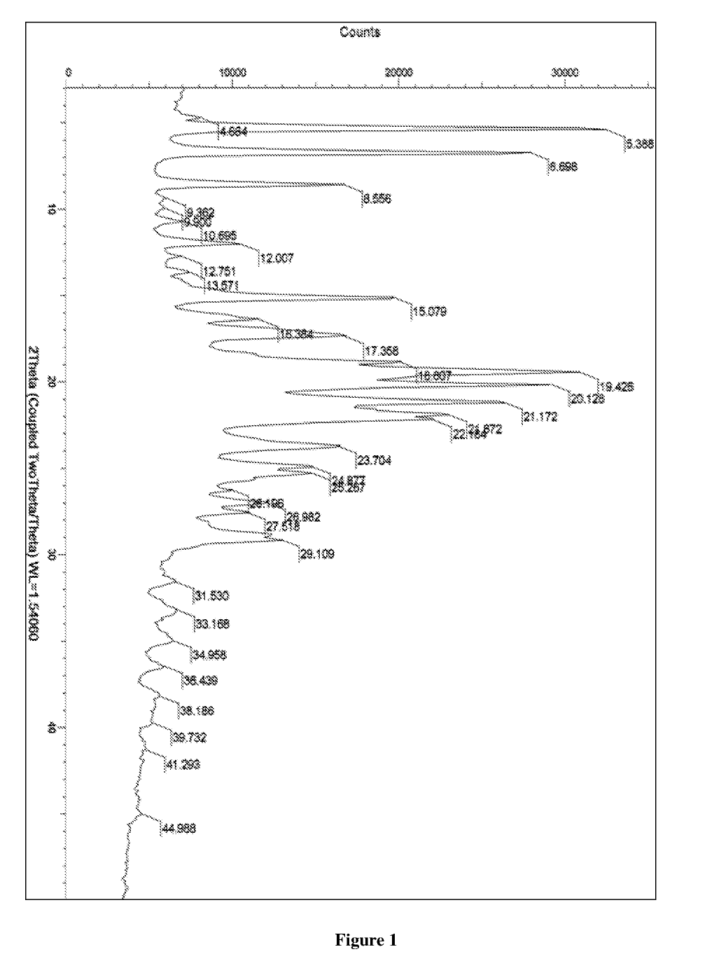

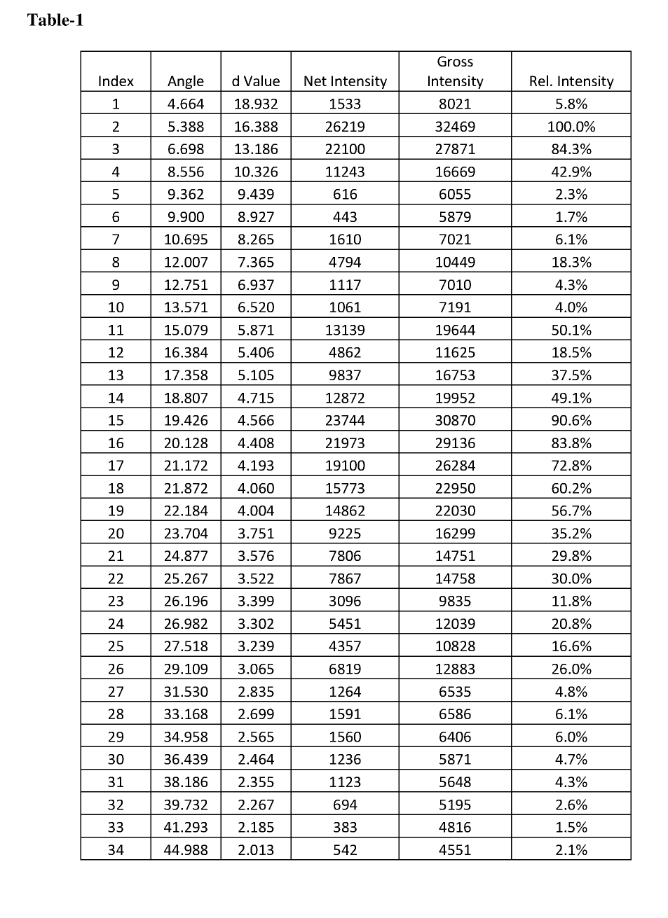

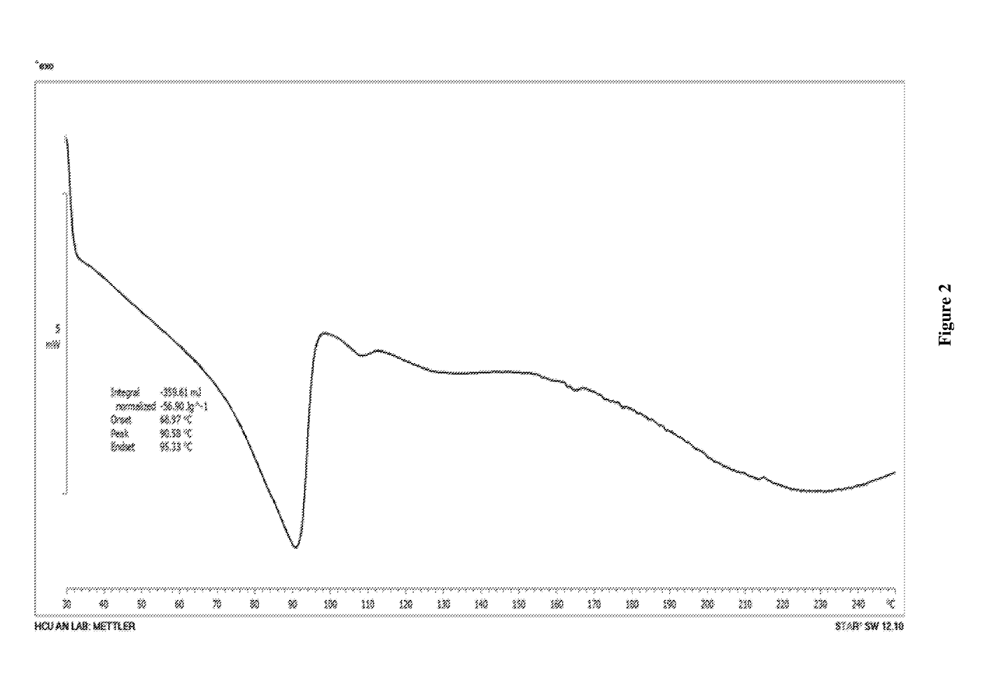

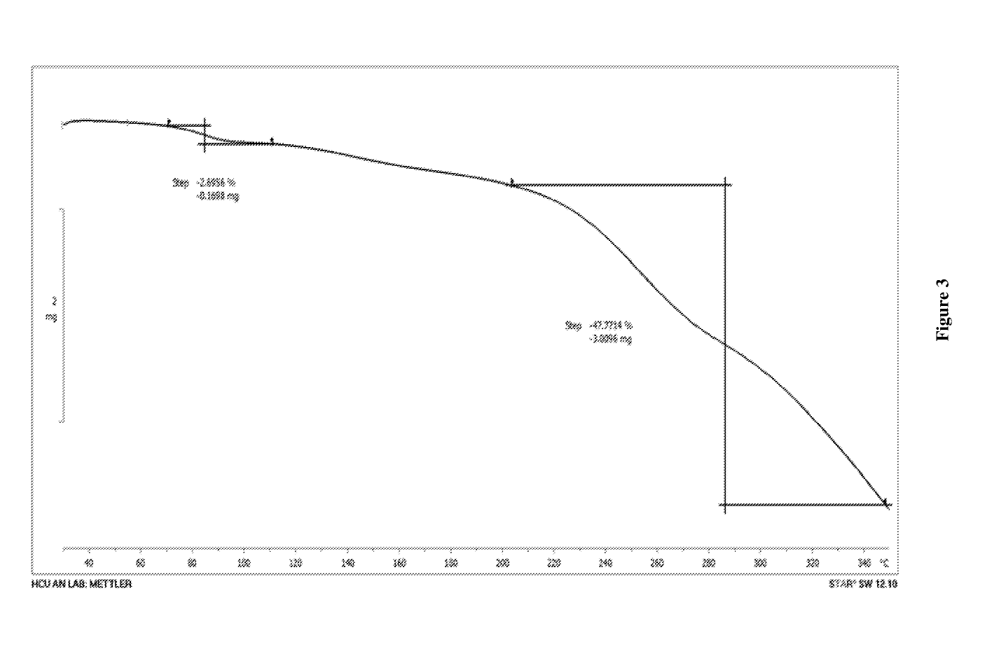

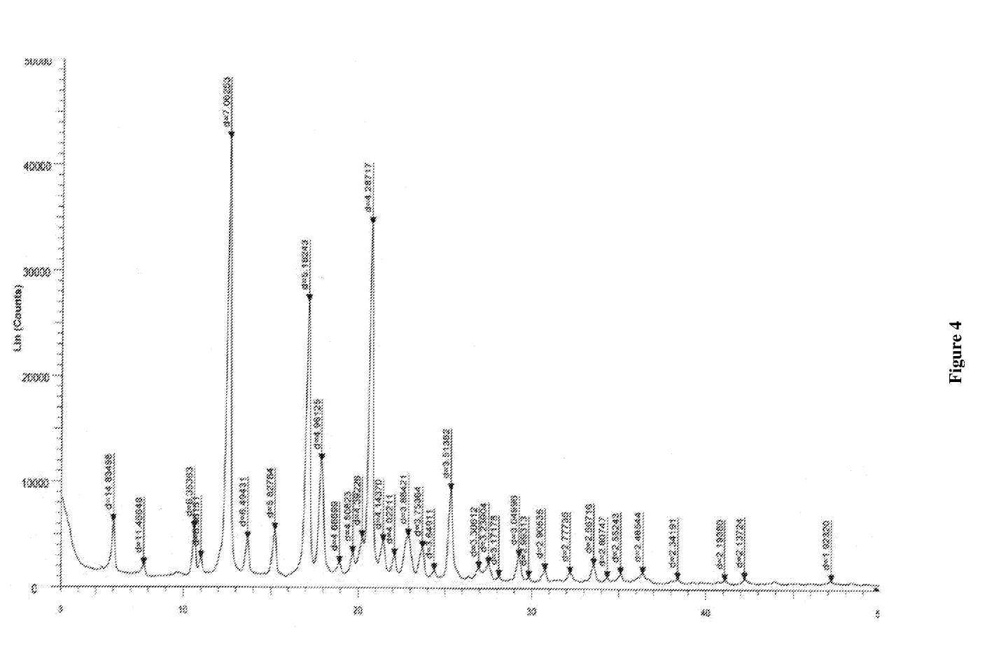

Characteristic Physico-Chemical Data of Crystalline
Form of the Compound of Formula VI

| | |
|---|---|
| Physical appearance: | Off-white to white solid |
| X-ray Powder Diffraction Pattern: | See FIG. 1 and Table 1 |
| DSC: | See FIG. 2 |
| TGA: | See FIG. 3 |

### Example—5: Preparation of (R)-1-(3-hydroxypropyl)-5-(2-(2-(2-(2,2,2-trifluoroethoxy) phenoxy)ethylamino)propyl)indoline-7-carbonitrile (VII)

(2R)-1-(7-cyano-1-(3-hydroxypropyl)-2,3-dihydro-1H-inden-5-yl)-N-(2-(2-(2,2,2-trifluoroethoxy)phenoxy)ethyl)propan-2-aminium (R)-4-acetamido-4-carboxybutanoate (VI) (10.5 g syrup) was stirred in water (75 mL) and DCM (75 mL), pH of the reaction mass was adjusted to 10-11 using Sodium hydroxide solution and layers were separated. DCM layer was concentrated to yield (R)-1-(3-hydroxypropyl)-5-(2-(2-(2-(2,2,2-trifluoroethoxy) phenoxy)ethylamino)propyl)indoline-7-carbonitrile (VII) (7.5 g).

### Example—6: Preparation of (R)-1-(1-(3-(benzoyloxy) propyl)-7-cyanoindolin-5-yl)-N-(2-(2-(2,2,2-trifluoroethoxy)phenoxy)ethyl)propan-2-aminium (S)-4-acetamido-4-carboxybutanoate (IV-a)

(R)-3-(7-cyano-5-(2-(2-(2-(2,2,2-trifluoroethoxy)phenoxy)ethylamino)propyl)indolin-1-yl)propyl benzoate (IV) (4.6 kg syrup) was dissolved in IPA (50 L), N-acetyl-L-glutamic acid was added (1.0 eq.,), reaction mass was stirred at 25° C. for 5 h then at 5 to 10° C. for 1 hr, solid was filtered, the wet cake was slurried with IPA at 55° C. to 75° C., stirred for 1 h at 25° C. & filtered. Wet material was dried at 40° C. under vacuum to (R)-1-(1-(3-(benzoyloxy) propyl)-7-cyanoindolin-5-yl)-N-(2-(2-(2,2,2-trifluoroethoxy)phenoxy)ethyl)propan-2-aminium (S)-4-acetamido-4-carboxybutanoate (IV-a) (4.73 kg).

1H-NMR (DMSO-d6) δ ppm: 1.0 (3H, d, J=6.1 Hz), 1.8 (1H, m), 1.9 (3H, s), 1.9 (1H, m), 2.1 (2H, t), 2.2-2.3 (2H, t), 2.4 (1H, t), 2.8 (1H, bd), 2.9 (2H, t), 3.2 (3H, bs), 3.6 (2H, t), 3.7 (2H, t, J=6.7 Hz), 4.2 (3H, bd), 4.4 (2H, bd), 4.7 (2H, q, J=9.8 Hz), 6.9 (1H, d), 7.0 (2H, t), 7.1 (3H, t), 7.5 (2H, t), 7.6 (1H, m, J=7.3 Hz), 7.9 (1H, d, J=7.9 Hz), 8.0 (2H, d), 9.1 (2H, bs).

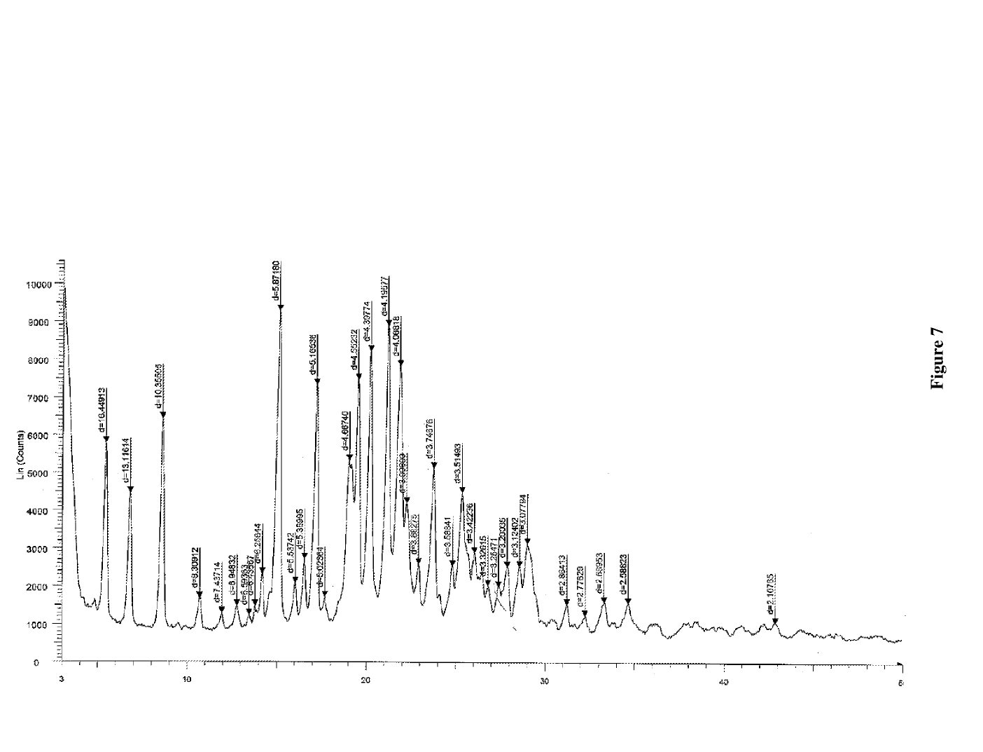

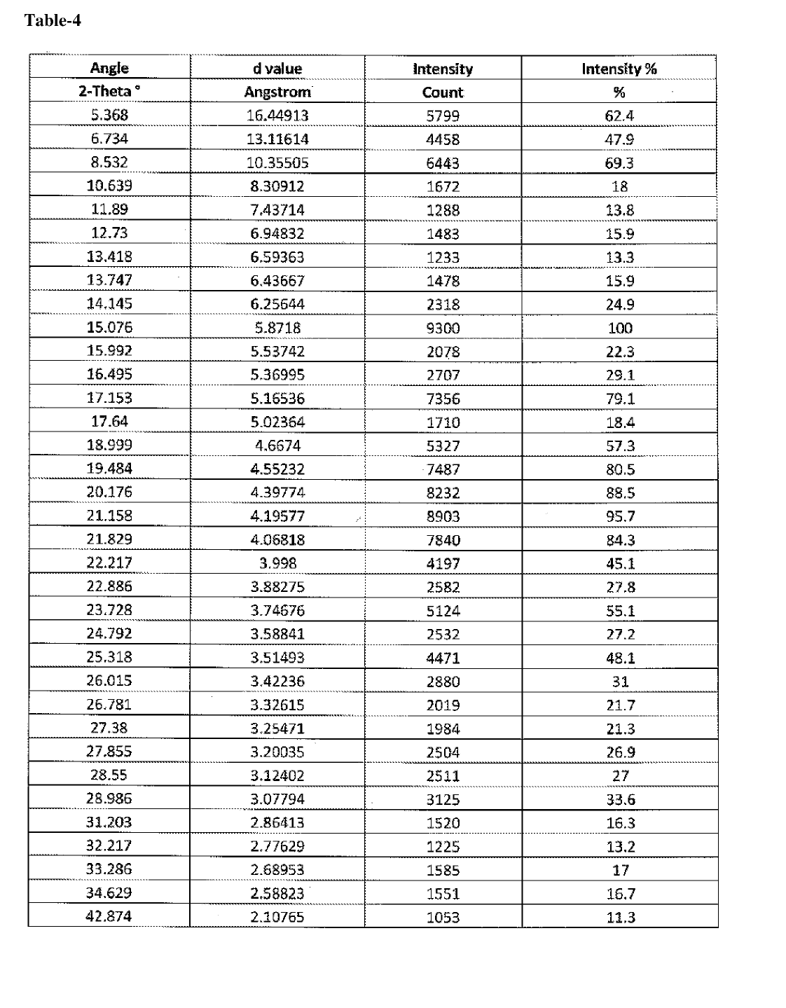

Characteristic Physico-Chemical Data of Crystalline
Polymorph Form of the Compound of Formula IV-a

| | |
|---|---|
| Melting point range: | 131.0 to 142.0° C. |
| Physical appearance: | Off-white to white solid |
| X-ray Powder Diffraction Pattern: | See FIG. 7 and Table 4 |

### Example—7: Preparation of (R)-3-(7-cyano-5-(2-(2-(2-(2,2,2-trifluoroethoxy)phenoxy) ethylamino)propyl)indolin-1-yl)propyl benzoate (IV-b)

(R)-1-(1-(3-(benzoyloxy)propyl)-7-cyanoindolin-5-yl)-N-(2-(2-(2,2,2-trifluoroethoxy) phenoxy)ethyl)propan-2-aminium (S)-4-acetamido-4-carboxybutanoate (IV-a)(4.0 kg) was stirred in water (20 L) and Ethyl acetate (20 L), pH of the reaction mass was adjusted to 10-11 using NaOH and layers separated. Ethyl acetate layer was concentrated to yield (R)-3-(7-cyano-5-(2-(2-(2-(2,2,2-trifluoroethoxy)phenoxy)ethylamino)propyl) indolin-1-yl)propyl benzoate (IV-b) (2.7 kg).

### Example—8: Preparation of (R)-1-(3-hydroxypropyl)-5-(2-(2-(2-(2,2,2-trifluoroethoxy) phenoxy)ethylamino)propyl)indoline-7-carbonitrile (VII)

(R)-1-(1-(3-(benzoyloxy)propyl)-7-cyanoindolin-5-yl)-N-(2-(2-(2,2,2-trifluoroethoxy) phenoxy)ethyl)propan-2-aminium(S)-4-acetamido-4-carboxybutanoate (IV-b) (2.7 kg) was dissolved in Methanol (19 L), Potassium hydroxide (4 eq.) solution (dissolved in water) was added to the reaction mass and stirred the reaction mass at 65° C. for reaction completion (around 5 hrs). The reaction mass was concentrated completely to remove Methanol, water (19 L) and Ethyl acetate (19 L), was added and stirred for 10 min. Layers were separated, organic layer was washed with bicarbonate solution and brine solution. The organic layer was concentrated completely to (R)-1-(3-hydroxypropyl)-5-(2-(2-(2-(2,2,2-trifluoroethoxy)phenoxy)ethylamino)propyl)indoline-7-carbonitrile (VII) (1.85 kg).

### Example—9: Preparation of (R)-1-(3-hydroxypropyl)-5-(2-(2-(2-(2,2,2-trifluoroethoxy) phenoxy)ethylamino)propyl)indoline-7-carboxamide (VIII)

(R)-1-(3-hydroxypropyl)-5-(2-(2-(2-(2,2,2-trifluoroethoxy) phenoxy)ethylamino)propyl) indoline-7-carbonitrile (VII) (1.85 kg) syrup was dissolved in DMSO, 5N Sodium hydroxide (1.85 L) solution was added at 5 to 10° C., 30% H₂O₂(845 mL) solution was added drop wise at 5 to 10° C., the reaction mass was stirred at room temperature for the reaction completion (around 5 hrs), sodium sulphite (0.95 kg in 36 L water) solution was added to the reaction mass followed by DCM (9 L), stirred for 10 min, the layers were separated. Toluene (13 L) was added to the organic layer and concentrated the organic layer at 54° C. to remove DCM completely then the Toluene layer was stirred at RT for 1 hr and MTBE (19 L) was added to the solid and stir it for 1 hr, filter the solid as silodosin crude.

### Example 10: Preparation of Silodosin Gamma Crystalline Form

IPA (2.6 L) and Toluene (21.6 L) was added to the crude solid (VIII) (1.8 kg) and stirred at 72° C. till dissolution approximately for 30 minutes, then stir the reaction mass at RT for 1 hr and add MTBE (9 L) and stirred at RT for 1 hr and the solid was filtered off to yield (R)-1-(3-hydroxypropyl)-5-(2-(2-(2-(2,2,2-trifluoroethoxy)phenoxy) ethyl amino)propyl)indoline-7-carboxamide (VIII) (1.4 kg). The wet cake was slurried with 9 L MTBE at 25° C., stirred for 2 h and filtered off, dried in vacuum drier under vacuum at 60° C.

### Example 11: Preparation of Silodosin Alpha Crystalline Form

Ethyl acetate (84 mL) was added to the crude solid (VIII) (7.0 g) and stirred at 54° C. for dissolution for 30 min then stir it at RT for 1 hr, filter the solid to yield (R)-1-(3-hydroxypropyl)-5-(2-(2-(2-(2,2,2-trifluoroethoxy)phenoxy)ethylamino)propyl)indoline-7-carboxamide (VIII) (5.5 g).

### Example—12: Preparation of (R)-1-(3-hydroxypropyl)-5-(2-(2-(2-(2,2,2-trifluoroethoxy) phenoxy)ethylamino)propyl)indoline-7-carboxamide (VIII) as crystalline beta (0) form

(R)-1-(3-hydroxypropyl)-5-(2-(2-(2-(2,2,2-trifluoroethoxy)phenoxy)ethyl amino) propyl) indoline-7-carbonitrile (VII) (7.0 g) syrup was dissolved in DMSO, 5N Sodium hydroxide (7 mL) solution was added at 5 to 10° C., 30% H₂O₂(3.2 mL) solution was added drop wise at 5 to 10° C., the reaction mass was stirred at room temperature for the reaction completion (around 5 hrs), sodium sulphite (3.6 g in 135 mL water) solution was added to the reaction mass followed by DCM (35 mL), stirred for 10 min, the layers were separated. Toluene (42 mL) was added to the organic layer and concentrated the organic layer at 54° C. to remove DCM completely and then the Toluene layer was stirred at 75±5° C. for 1 hr and Methyl Tertiary Butyl Ether (84 mL) was added to the clear solution and stirred it for 30 min, cooled the mass to 65±5° C., seed with 1% beta silodosin and stir at RT for 1 hr, the solid was filtered as silodosin crystalline beta (β) form.

### Example 13: Preparation of Silodosin Beta Crystalline Form from Gamma Crystalline Form

Silodosin (gamma) 40 g was dissolved in 280 mL Toluene at 75° C. till clear solution observed. 480 mL of MTBE was added and stirred for 1 h at the same temperature, cooled to 60° C. and seeded with 1% Silodosin beta form. Stirring was continued for 1 h at RT, solid was filtered off to yield (R)-1-(3-hydroxypropyl)-5-(2-(2-(2-(2,2,2-trifluoroethoxy)phenoxy)ethyl amino)propyl)indoline-7-carboxamide (VIII) (30 g) beta form.

1. A method for preparation of 1-(3-hydroxypropyl)-5-[(2R)-2-({2-[2-(2,2,2-trifluoroethoxy)phenoxy]ethyl}amino)-propyl]-2,3-dihydro-1H-indole-7-carboxamide of the structural formula (VIII): 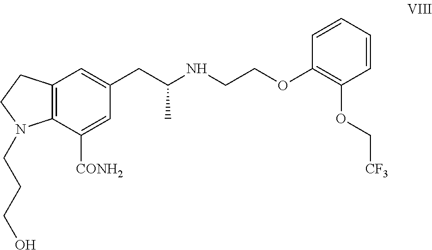 [SMILES: C[C@H](Cc1cc2c(c(C(N)=O)c1)N(CCCO)CC2)NCCOc1ccccc1OCC(F)(F)F]
comprising converting 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile of the structural formula (V) 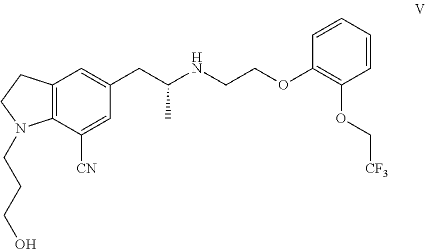 [SMILES: C[C@H](Cc1cc(C#N)c2c(c1)CCN2CCCO)NCCOc1ccccc1OCC(F)(F)F]
to a carboxylate salt, subsequently neutralizing and hydrolyzing to yield 1-(3-hydroxypropyl)-5-[(2R)-2-({2-[2-(2,2,2-trifluoroethoxy)phenoxy]ethyl}amino)-propyl]-2,3-dihydro-1H-indole-7-carboxamide of the structural formula (VIII).

2. A method for preparation as claimed in claim 1, comprises of 1-(3-hydroxypropyl)-5-[(2R)-2-({2-[2-(2,2,2-trifluoroethoxy)phenoxy]ethyl}amino)-propyl]-2,3-dihydro-1H-indole-7-carboxamide of the structural formula (VIII): 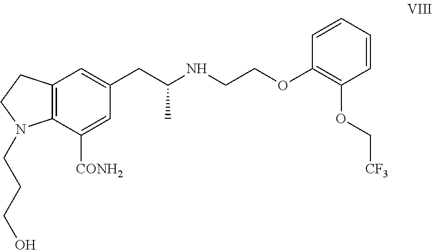 [SMILES: C[C@H](Cc1cc2c(c(C(N)=O)c1)N(CCCO)CC2)NCCOc1ccccc1OCC(F)(F)F]
mixing 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile of the structural formula (V): 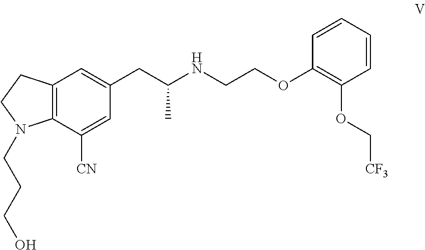 [SMILES: C[C@H](Cc1cc(C#N)c2c(c1)CCN2CCCO)NCCOc1ccccc1OCC(F)(F)F]
with N-acetyl glutamic acid to yield 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile N-acetyl mono glutamate of the structural formula (VI); 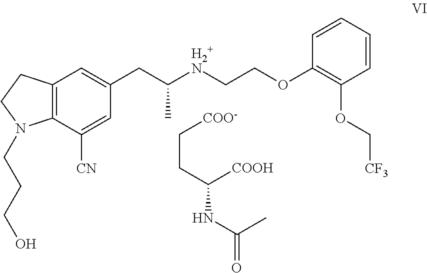
subsequently neutralizing the glutamate and hydrolyzing to yield 1-(3-hydroxypropyl)-5-[(2R)-2-({2-[2-(2,2,2-trifluoroethoxy)phenoxy]ethyl}amino)-propyl]-2,3-dihydro-1H-indole-7-carboxamide of the structural formula (VIII).

3. A method for preparation as claimed in claim 1, which comprises neutralising the 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile N-acetyl mono glutamate to 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile of the structural formula (VII).

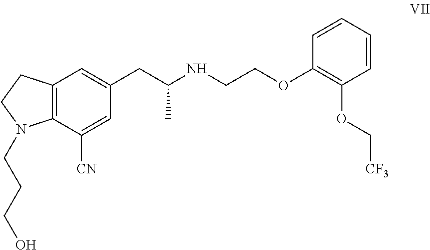 [SMILES: C[C@H](Cc1cc(C#N)c2c(c1)CCN2CCCO)NCCOc1ccccc1OCC(F)(F)F]

4. A one pot process for the preparation of 1-(3-hydroxypropyl)-5-[(2R)-2-({2-[2-(2,2,2-trifluoroethoxy)phenoxy]ethyl}amino)propyl]-2,3-dihydro-1H-indole-7-carboxamide of the structural formula (VIII) as crystalline gamma form comprising of: 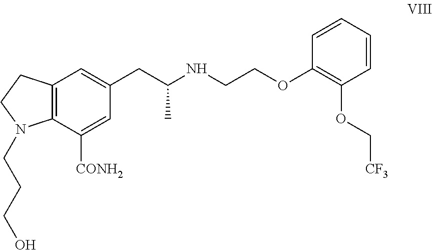 [SMILES: C[C@H](Cc1cc2c(c(C(N)=O)c1)N(CCCO)CC2)NCCOc1ccccc1OCC(F)(F)F]
a) Reacting 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile (VII) with an alkali solution 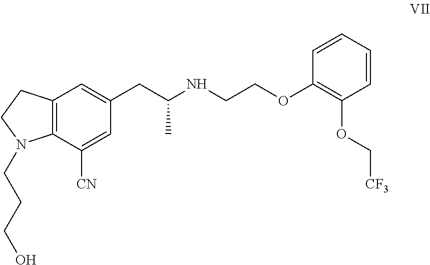 [SMILES: C[C@H](Cc1cc(C#N)c2c(c1)CCN2CCCO)NCCOc1ccccc1OCC(F)(F)F]
in presence of an oxidizing agent at lower temperature
b) Quenching the reaction mass of step a) with sodium sulphite solution
c) Diluting the reaction mass of step b) with a halogenated aliphatic hydrocarbon solvent and removal of aqueous layer.
d) Diluting the organic layer of step c) with an aromatic hydrocarbon solvent and removal of halogenated aliphatic hydrocarbon.
e) Cooling the solution of step d) to room temperature
f) Addition of a C1 to C6 aliphatic ether to the aromatic hydrocarbon solution of step e)
g) Filtering silodosin as gamma crystalline form.

5. A one pot process for the preparation of 1-(3-hydroxypropyl)-5-[(2R)-2-({2-[2-(2,2,2-trifluoroethoxy)phenoxy]ethyl}amino)propyl]-2,3-dihydro-1H-indole-7-carboxamide of the structural formula (VIII) as crystalline gamma form as claimed in claim 4 comprising of: 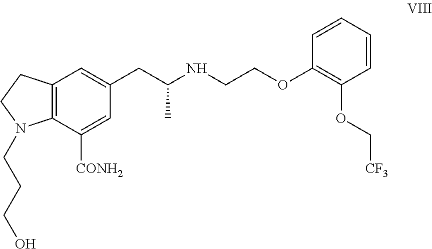 [SMILES: C[C@H](Cc1cc2c(c(C(N)=O)c1)N(CCCO)CC2)NCCOc1ccccc1OCC(F)(F)F]
a) Reacting 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile (VII) with aqueous sodium hydroxide solution 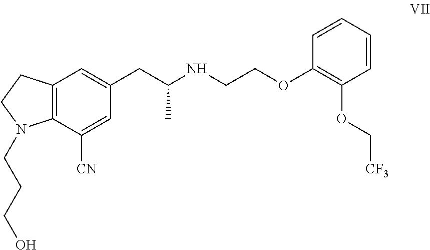 [SMILES: C[C@H](Cc1cc(C#N)c2c(c1)CCN2CCCO)NCCOc1ccccc1OCC(F)(F)F]
in presence of hydrogen peroxide at 5 to 10° C.
b) Quenching the reaction mass of step a) with sodium sulphite solution
c) Diluting the reaction mass of step b) with dichloromethane and removal of aqueous layer
d) Diluting the organic layer of step c) with toluene and removal of dichloromethane
e) Cooling the solution of step d) to room temperature
f) Addition of a methyl tertiary butyl ether to the solution of step e)
g) Filtering silodosin as gamma crystalline form.

6. A process for preparation of Gamma crystalline form of silodosin comprising dissolving silodosin in a mixture of C-1 to C-6 alcohol and an aromatic hydrocarbon optionally at an elevated temperature followed by addition of an anti-solvent preferably an ether at lower temperature followed by stirring and filtration

7. A method of preparation as claimed in claim 6 wherein C-1 to C-6 alcohol is isopropanol, methanol, ethanol and propanol

8. A method of preparation as claimed in claim 7 wherein C-1 to C-6 alcohol is isopropanol

9. A method of preparation as claimed in claim 6 wherein an aromatic hydrocarbon hydrocarbon solvent is toluene, benzene, ethyl benzene and xylene

10. A method of preparation as claimed in claim 9 wherein hydrocarbon solvent is toluene

11. A method of preparation as claimed in claim 6 wherein ether is methyl tertiary butyl ether, diethyl ether, methyl ethyl ether and methyl phenyl ether

12. A method of preparation as claimed in claim 11 wherein ether is methyl tertiary butyl ether

13. A process for the preparation of gamma crystalline form of silodosin according to claim 6, wherein residual toluene content is less than 890 ppm as obtained by the process comprising the steps of;
a) Slurrying gamma crystalline form of silodosin in an ether, preferably methyl tertiary butyl ether at ambient temperature or at lower temperature followed by stirring and filtration
b) Drying the resulting compound
Isolating polymorphic form gamma of silodosin having toluene content less than 890 ppm.

14. 1-(3-hydroxy propyl)-5-[(2R)-2-({2-(2,2,2-trifluoroethoxy)phenoxy]ethyl}amino)-propyl]2,3-dihydro-1H-indole-7-carbonitrile N-acetyl mono glutamate.

15. A crystalline form of 1-(3-hydroxy propyl)-5-[(2R)-2-({2-(2,2,2-trifluoroethoxy)phenoxy]ethyl}amino)-propyl]2,3-dihydro-1H-indole-7-carbonitrile N-acetyl mono glutamate characterized by a XRD pattern with strong peaks at 5.38, 6.69, 8.55, 12.0, 15.08, 16.38, 17.35, 18.80, 19.42, 20.12, 21.17, 21.87, 22.18, 23.7, 24.87, 25.26, 26.19, 26.98, 27.51, 29.10±0.2 degrees 2θ.

16. A crystalline form of 1-(3-hydroxy propyl)-5-[(2R)-2-({2-(2,2,2-trifluoroethoxy)phenoxy]ethyl}amino)-propyl]2,3-dihydro-1H-indole-7-carbonitrile N-acetyl mono glutamate as claimed in claim 15 characterized by a XRD pattern as depicted in FIG. 1

17. A one pot process for the preparation of 1-(3-hydroxypropyl)-5-[(2R)-2-({2-[2-(2,2,2-trifluoroethoxy)phenoxy]ethyl}amino)propyl]-2,3-dihydro-1H-indole-7-carboxamide of the structural formula (VIII) as crystalline beta form comprising of: 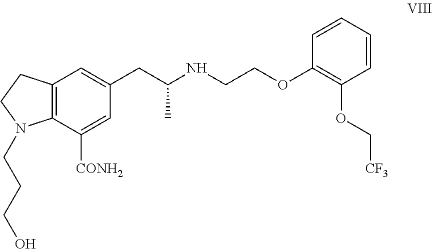 [SMILES: C[C@H](Cc1cc2c(c(C(N)=O)c1)N(CCCO)CC2)NCCOc1ccccc1OCC(F)(F)F]
a) Reacting 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile (VII) with aqueous alkali solution 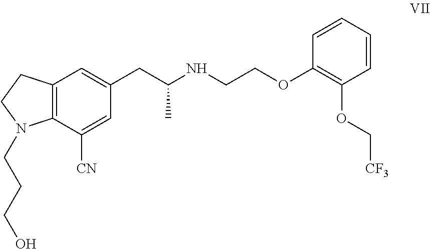 [SMILES: C[C@H](Cc1cc(C#N)c2c(c1)CCN2CCCO)NCCOc1ccccc1OCC(F)(F)F]
in presence of an oxidizing agent at lower temperature
b) Quenching the reaction mass with sodium sulphite solution
c) Diluting the reaction mass with a halogenated aliphatic hydrocarbon solvent and removal of aqueous layer.
d) Diluting the organic layer with an aromatic hydrocarbon solvent and removal of halogenated aliphatic hydrocarbon.
e) Addition of a C1 to C6 aliphatic ether to the aromatic hydrocarbon solution at elevated temperature.
f) Partially cooling the solution of step e), seeding with crystalline beta silodosin.
g) Slowly cooling to room temperature and filtering silodosin as beta crystalline form.

18. A one pot process for the preparation of 1-(3-hydroxypropyl)-5-[(2R)-2-({2-[2-(2,2,2-trifluoroethoxy)phenoxy]ethyl}amino)propyl]-2,3-dihydro-1H-indole-7-carboxamide of the structural formula (VIII) as crystalline beta form as claimed in claim 17, comprising of: 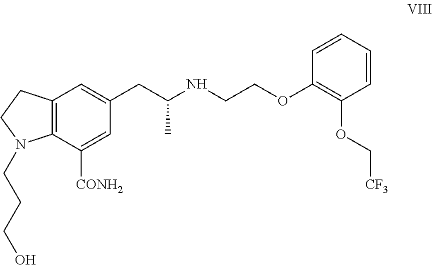 [SMILES: C[C@H](Cc1cc2c(c(C(N)=O)c1)N(CCCO)CC2)NCCOc1ccccc1OCC(F)(F)F]
a) Reacting 1-(3-hydroxypropyl)-5-[(2R)-2-({2-[2-(2,2,2-trifluoroethoxy)phenoxy]ethyl}amino)propyl]-2,3-dihydro-1H-indole-7-carbonitrile (VII) with aqueous sodium hydroxide solution 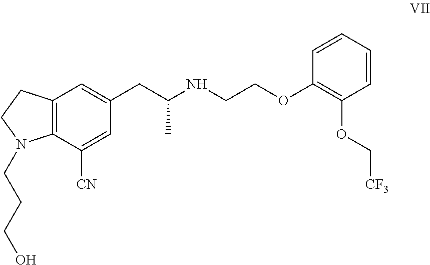 [SMILES: C[C@H](Cc1cc(C#N)c2c(c1)CCN2CCCO)NCCOc1ccccc1OCC(F)(F)F]
in presence of hydrogen peroxide at 5 to 10° C.
b) Quenching the reaction mass with sodium sulphite solution
c) Diluting the reaction mass with dichloromethane and removal of aqueous layer
d) Diluting the organic layer with toluene and removal of dichloromethane
e) Addition of methyl tertiary butyl ether to the organic layer of step d) at 75 to 80° C.
f) Partially cooling the solution of step e) to 65 to 70° C., seeding with crystalline beta silodosin
g) Slowly cooling to room temperature and filtering silodosin as beta crystalline form.

19. A process for the preparation of silodosin crystalline beta form from silodosin crystalline gamma form comprising steps of:
a) Dissolving the silodosin crystalline gamma form in an aromatic hydrocarbon solvent at an elevated temperature
b) Addition of a C1 to C6 aliphatic ether solvent to the aromatic hydrocarbon solution of step a) at elevated temperature
c) Partially cooling the solution of step b), seeding with crystalline beta silodosin
d) Slowly cooling to room temperature and filtering silodosin as beta crystalline form.

20. A process for the preparation of silodosin crystalline beta form from silodosin crystalline gamma form as claimed in claim 19, comprising steps of:
a) Dissolving silodosin crystalline gamma form in toluene at 75° C.
b) Addition of a methyl tertiary butyl ether to solution of step a) at 75° C.
c) Partially cooling the solution of step b) to 60° C., seeding with crystalline beta silodosin.
d) Slowly cooling to room temperature and filtering silodosin as beta crystalline form.
